SEQUENCE LISTING (13 sequences)

SEQ ID NO: 1 - DNA, 14 bp, Artificial sequence
  antisense oligonucleotides
gtgtgaacttggct

SEQ ID NO: 2 - DNA, 16 bp, Artificial sequence
  antisense oligonucleotides
tttgagagttggagga

SEQ ID NO: 3 - DNA, 16 bp, Artificial sequence
  antisense oligonucleotides
tcatactatatgacag

SEQ ID NO: 4 - DNA, 22 bp, Artificial sequence
  primer
cctctagatgtgtaagggtagt

SEQ ID NO: 5 - DNA, 21 bp, Artificial sequence
  primer
ttgagttgcatagttgaggaa

SEQ ID NO: 6 - DNA, 20 bp, Artificial sequence
  primer
tgggaacaagagggcatctg

SEQ ID NO: 7 - DNA, 22 bp, Artificial sequence
  primer
ccaccactgcatcaaattcatg

SEQ ID NO: 8 - DNA, 21 bp, Artificial sequence
  primer
tgacactggcaaaacaatgca

SEQ ID NO: 9 - DNA, 21 bp, Artificial sequence
  primer
ggtccttttcaccagcaagct

SEQ ID NO: 10 - DNA, 19 bp, Artificial sequence
  primer
atttgggtcgcggttcttg

SEQ ID NO: 11 - DNA, 21 bp, Artificial sequence
  primer
tgccttgacattctcgatggt

SEQ ID NO: 12 - DNA, 20 bp, Artificial sequence
  primer
cacgaaccacggcactgatt

SEQ ID NO: 13 - DNA, 22 bp, Artificial sequence
  primer
ttttcttgctgccagtctggac

(19) United States
(12) Patent Application Publication
(10) Pub. No.: US 2022/0218734 A1
(43) Pub. Date: Jul. 14, 2022

(54) COMBINATIONS OF THERAPEUTIC AGENTS FOR TREATING UVEAL MELANOMA

(71) Applicant: Universiteit Gent, Gent (BE)

(72) Inventors: Pieter Mestdagh, Brugge (BE); Shanna Dewaele, Bissegem (BE)

(21) Appl. No.: 17/611,828

(22) PCT Filed: May 25, 2020
(86) PCT No.: PCT/EP2020/064430

§ 371 Date: Nov. 16, 2021

(30) Foreign Application Priority Data
May 29, 2019 (EP) 19177294.6

(51) Int. Cl. A61K 31/7105; A61K 38/46; A61K 31/5377; A61K 31/167; A61K 31/426; A61K 31/567; A61K 31/47; A61P 35/00

(52) U.S. Cl. CPC A61K 31/7105; A61K 38/465; A61K 31/5377; A61P 35/00; A61K 31/426; A61K 31/567; A61K 31/47; A61K 31/167

(57) ABSTRACT

A combination of therapeutic compounds for treating cancer. More specifically, the present discloses that inhibition of the long non-coding RNA known as SAMMSON in combination with at least one of FDA-approved, anti-cancer compounds results in killing of uveal melanoma cells in a synergistic manner.

### CROSS-REFERENCE TO RELATED APPLICATIONS

This application is a national phase entry under 35 U.S.C. § 371 of International Patent Application PCT/EP2020/064430, filed May 25, 2020, designating the United States of America and published in English as International Patent Publication WO 2020/239685 A1 on Dec. 3, 2020, which claims the benefit under Article 8 of the Patent Cooperation Treaty to European Patent Application Serial No. 19177294.6, filed May 29, 2019.

### TECHNICAL FIELD

This application relates to a combination of therapeutic compounds for treating cancer. More specifically, disclosed is that inhibition of the long non-coding RNA known as SAMMSON in combination with at least one of FDA-approved, anti-cancer compounds results in killing of uveal melanoma cells in a synergistic manner.

### BACKGROUND

Uveal melanoma (UM) is the most common primary intraocular malignancy in adults, with an incidence of 5-7.4 cases per million annually¹⁻³. This rare type of melanoma is a genetically and biologically distinct type of melanoma that arises from choroidal melanocytes, which are melanocytes of the choroidal plexus, ciliary body and iris of the eye. This type of melanoma does not harbor any of the hallmark mutations detected in skin melanoma, such as BRAF, NRAS or KIT, but instead shows mutations in GNAQ or GNA11⁴. Current treatments consist of radiotherapy and enucleation, but despite of the progress in local therapy, no significant progress in overall survival has been achieved. The main cause of death of UM patients is the metastatic dissemination, mainly to the liver, that appears in about 50% of the patients. Currently, no effective treatment modality is available for patients with metastatic uveal melanoma, which results in a median survival time for UM patients diagnosed with metastasis of 6-12 months⁵. Because of the high unmet need, identifying novel therapeutic targets for uveal melanoma are crucial.

The long non-coding RNA (lncRNA) Survival Associated Mitochondrial Melanoma Specific Oncogenic Non-coding RNA (SAMMSON), also known as LINC01212, is recently described as a lineage survival oncogene in skin melanoma. PAN cancer RNA sequencing data from more than 10 000 primary tumors representing 32 cancer types (The Cancer Genome Atlas, TCGA) showed the highest and most consistent expression of SAMMSON in skin melanoma tumors (SKCM, >90%), but also revealed a lower but sustained upregulation of SAMMSON in uveal melanoma tumors (UVM, >80%), while no expression can be observed in primary melanocytes, normal adult tissues and most other cancer types.

Since lncRNA expression is strikingly cell type- and tissue-specific, this opens the opportunity for targeted therapy⁶. Therapeutic nucleic acid-based approaches like siRNA and antisense oligonucleotides (ASO) hold enormous potential to target lncRNAs. Therapeutic antisense technology research has advanced dramatically over the last decades to make the development of ASO-based drugs possible. The chemical modifications result in overcoming degradation by endogenous nucleases, the possibility to cross vascular endothelium, extracellular matrix and cell membranes in order to reach the intracellular target and to ensure target recognition inside cells without offside effects⁷. These efforts have led to the US Food and Drug Administration (FDA) approval of several ASOs to treat diseases such as spinal muscular atrophy (SMA), Myotonic Dystrophy type I (DM1), etc^{7,8}.

### BRIEF DESCRIPTION OF THE DRAWINGS

FIG. 1 Schematic representation of drug screening procedure. An FDA-approved library consisting of 2,911 compounds was screened in combination with LNA NTC or LNA ASO 3 in UM cell line 92.1 in 1 replicate. 384 compounds were selected for a confirmation screen in 92.1 in 4 replicates. Based on viability results 48 h after treatment, 18 compounds were further extensively validated in 2 UM cell lines (92.1 and OMM1) resulting in the final selection of 2 compounds for in vivo assessment of synergistic effects.

FIG. 2 In vitro validation of SAMMSON inhibition in combination with compounds from an FDA approved library. A. Bscores as a viability measure in UM cell line 92.1 48 h after transfection with LNA NTC or LNA ASO 3 (35 nM) and compound treatment (10 μM). A black dot represents the Bscore of a compound in the ASO condition, a grey dot represent the Bscore difference (Bscore NTC−Bscore ASO) of the same compound. B, C. Viability results 48 h after treatment of UM cell line 92.1 treated with either LNA NTC (35 nM), LNA ASO 3 (35 nM), LNA NTC (35 nM)+compound (10 μM) and LNA ASO 3 (35 nM)+compound (10 Values are relative to LNA NTC treated cells (black horizontal line). The horizontal black dashed line represents viability effect of LNA ASO 3 treated cells. Black and grey dots are showing the median viability effects of each compound when combined with LNA NTC and LNA ASO 3, respectively. Error bars represent the median±95% confidence interval (CI) of n=4. C. Detailed viability results of the 18 selected compounds from the confirmation screen. D. Excess over Bliss scores were calculated to investigate synergism in 2 UM cell lines (92.1 and OMM1) with 2 different read outs (viability and confluence) 72 h after treatment of the cells with the compounds and LNA NTC or LNA ASO 3 (50 nM). Excess over Bliss scores>0 indicate synergism, <0 indicate antagonism. E-K. Viability (72 h time point) and relative confluence effects of UM cell lines 92.1 and OMM1 after transfection of LNA NTC or LNA ASO 3 (50 nM) with varying concentrations of the 7 selected compounds: Amsacrine (E), Clioquinol (F), GDC-0349 (G), Mifepristone (H), CI-994 (I), Entinostat (J) and INH-6 (K). The real-time effect on confluence is shown for a fixed compound concentration. Confluence results are relative to the first 4 measured data points.

FIG. 3 mTOR inhibition enhances SAMMSON ASO activity in vitro. A. Heat map representation of differentially expressed genes in OMM1 and 92.1 cells 24 h after treatment with NTC ASO (50 nM), ASO 3 (50 nM), GDC-0349 (0.625 μM) or combination of ASO 3 (50 nM) and GDC-0349 (0.625 B-D. Mitochondrial stress test seahorse profiles of OMM1 cells treated with LNA NTC (50 nM) and LNA ASO 3 (50 nM) (B), GDC-0349 (0.625 (C) or LNA ASO 3 (50 nM)+GDC-0349 (0.625 μM) (D). Error bars represent mean±SD of n=3. E. Spare respiratory capacity of OMM1 cells calculated as the difference between maximal and basal oxygen consumption rates (OCR). Relative OCR values are relative compared to NTC values. F. Western blot analysis for 4EBP1, phospho-4EBP1, rpS6, phospho-rpS6 after treatment with LNA NTC ASO or LNA ASO 3 (50 nM) and subsequent treatment with mTOR inhibitor GDC-0349 (0.625 μM or 1.25 μM) for 24 h in UM cell line 92.1. G. Quantification of protein synthesis measured by the incorporated puromycin signal on Western blot, in 92.1 and OMM1 cells. Cells were treated as described in A for 24 h and subsequently treated with puromycin for 10 min. H. Viability results and relative SAMMSON expression of OMM1 cells treated as described in A for 72 h and 48 h, respectively. Results are scaled to NTC; error bars represent mean±95% CI of n=4 (viability) and n=6 (expression). *** p≤0.001, **** p≤0.0001 determined by One-way ANOVA with Tukey's multiple comparisons test.

### DETAILED DESCRIPTION

Described herein is that SAMMSON is crucial for the survival of UM cells. ASOs containing a gapmer configuration with LNA modification were used to knock down SAMMSON and are further mentioned as LNA ASO or LNA gapmer. Knock down of SAMMSON using two independent LNA ASOs results in a decreased viability with induction of apoptosis, irrespective of the mutational status of the UM cell line. Subcutaneous administration of SAMMSON inhibiting LNA ASOs in a patient derived xenograft (PDX) UM mouse model results in a slower progression of the tumor. More importantly, an unbiased screening of an FDA approved compound library, consisting of 2911 compounds, in combination with a SAMMSON targeting LNA ASO results in the identification of ASO-compound combinations that significantly reduce cell viability in two well established UM cell lines. Indeed, Excess over Bliss score calculations demonstrated that combining an SAMMSON inhibiting ASO (LNA ASO 3) with at least one of the following 7 compounds amsacrine, GDC-0349, entinostat, INH-6, mifepristone, CI-994 or clioquinol act in a synergistic manner.

Together, these results highlight that SAMMSON targeting inhibitors such as ASOs in combination with at least one of the compounds that is selected from a topoisomerase II inhibitor such as amsacrine, a mammalian target of rapamycin (mTOR) inhibitor such as GDC-0349, a Histone deacetylase (HDAC) inhibitor such as entinostat and/or CI-994, an inhibitor of highly expressed in cancer 1 (Hec1) and its regulator Nek2, such as INH-6, a progesterone receptor antagonist such as mifepristone, and/or a zinc ionophore such as clioquinol can be used as a novel therapeutic option for the treatment of UM.

Therefore, the disclosure relates in first instance to a composition comprising: a. an inhibitor of functional expression of survival associated mitochondrial melanoma specific oncogene non-coding RNA (SAMMSON), which targets the SAMMSON gene or transcript directly by way of sequence complementarity and which is selected from a gapmer, a shRNA, a siRNA, an antisense RNA, a TALEN or a Zinc-finger nuclease, and b. at least one compound that is selected from a topoisomerase II inhibitor such as amsacrine, a mammalian target of rapamycin (mTOR) inhibitor such as GDC-0349, a HDAC inhibitor such as entinostat and/or CI-994, a Hec1/Nek2 inhibitor such as INH-6, a progesterone receptor antagonist such as mifepristone, and/or a zinc ionophore such as clioquinol for use to treat uveal melanoma.

The disclosure further relates to a composition for use as described above wherein the topoisomerase II inhibitor is amsacrine and/or wherein the mTOR inhibitor is GDC-0349 and/or wherein the HDAC inhibitor is entinostat and/or CI-994, and/or wherein the Hec1/Nek2 inhibitor is INH-6, and/or wherein the progesterone receptor antagonist is mifepristone, and/or wherein the zinc ionophore is clioquinol.

The disclosure further relates to a composition for use as described above wherein the inhibitors of functional expression of survival associated mitochondrial melanoma specific oncogene non-coding RNA (SAMMSON), which are locked nucleic acid antisense oligonucleotides (LNA gapmers) have the following nucleic acid sequences:

(LNA ASO 3 (SEQ ID NO: 1))
GTGTGAACTTGGCT
and
(LNA ASO 11 (SEQ ID NO: 2))
TTTGAGAGTTGGAGGA.

The disclosure also relates to a method to treat uveal melanoma comprising administering a pharmaceutically effective amount of a composition comprising: a. an inhibitor of functional expression of survival associated mitochondrial melanoma specific oncogene non-coding RNA (SAMMSON), which targets the SAMMSON gene or transcript directly by way of sequence complementarity and which is selected from a gapmer, a shRNA, a siRNA, an antisense RNA, a TALEN or a Zinc-finger nuclease, and b. at least one compound, which is selected from a topoisomerase II inhibitor such as amsacrine, a mammalian target of rapamycin (mTOR) inhibitor such as GDC-0349, a HDAC inhibitor such as entinostat and/or CI-994, a Hec1/Nek2 inhibitor such as INH-6, a progesterone receptor antagonist such as mifepristone, and/or a zinc ionophore such as clioquinol to a person in need thereof.

The disclosure further relates to a method to treat as described above to wherein the topoisomerase II inhibitor is amsacrine and/or wherein the mTOR inhibitor is GDC-0349 and/or wherein the HDAC inhibitor is entinostat and/or CI-994, and/or wherein the Hec1/Nek2 inhibitor is INH-6, and/or wherein the progesterone receptor antagonist is mifepristone, and/or wherein the zinc ionophore is clioquinol.

The disclosure further relates to a method to treat as described above to wherein the inhibitors of functional expression of survival associated mitochondrial melanoma specific oncogene non-coding RNA (SAMMSON), which are locked nucleic acid antisense oligonucleotides (LNA gapmers) have the following nucleic acid sequences:

(LNA ASO 3 (SEQ ID NO: 1))
GTGTGAACTTGGCT
and
(LNA ASO 11 (SEQ ID NO: 2)
TTTGAGAGTTGGAGGA.

The term Survival Associated Mitochondrial Melanoma Specific Oncogenic Non-coding RNA (SAMMSON) or “LINC01212” or “survival associated mitochondrial melanoma specific oncogenic non-coding RNA,” or “RP11-460N16.1” as used herein refers to the gene with accession number ENSG00000240405 in Ensembl (release 96), as well as the mRNA that is transcribed from the gene. It can also be identified with Gene ID: 101927152 or the human gene nomenclature identifier HGNC: 49644. As it is a non-protein coding gene, there is no protein product. In humans, the gene is located on the short arm of chromosome 3, from position 69,999,733 to 70,518,064. According to Ensembl and LNCipedia, the gene has 27 annotated transcripts (or splice variants) (Ensembl release 96) (Table 1) and 42 annotated transcripts (or splice variants) (LNCipedia version 5.2) (Table 2), respectively. All transcripts are lincRNAs (large intergenic non-coding RNAs).

TABLE 1
Annotated SAMMSON transcripts in Ensembl

| Name | Transcript ID | bp |
|---|---|---|
| SAMMSON-201 | ENST00000483525.2 | 2055 |
| SAMMSON-202 | ENST00000488861.6 | 1433 |
| SAMMSON-203 | ENST00000641005.1 | 2168 |
| SAMMSON-204 | ENST00000641014.1 | 1355 |
| SAMMSON-205 | ENST00000641043.1 | 1377 |
| SAMMSON-206 | ENST00000641053.1 | 1516 |
| SAMMSON-207 | ENST00000641128.1 | 1169 |
| SAMMSON-208 | ENST00000641194.1 | 868 |
| SAMMSON-209 | ENST00000641222.1 | 1100 |
| SAMMSON-210 | ENST00000641260.1 | 1308 |
| SAMMSON-211 | ENST00000641286.1 | 1645 |
| SAMMSON-212 | ENST00000641295.1 | 1487 |
| SAMMSON-213 | ENST00000641336.1 | 1521 |
| SAMMSON-214 | ENST00000641395.1 | 1264 |
| SAMMSON-215 | ENST00000641431.1 | 1046 |
| SAMMSON-216 | ENST00000641457.1 | 1110 |
| SAMMSON-217 | ENST00000641532.1 | 1657 |
| SAMMSON-218 | ENST00000641601.1 | 1847 |
| SAMMSON-219 | ENST00000641635.1 | 1162 |
| SAMMSON-220 | ENST00000641648.1 | 1207 |
| SAMMSON-221 | ENST00000641715.1 | 1546 |
| SAMMSON-222 | ENST00000641901.1 | 2727 |
| SAMMSON-223 | ENST00000641923.1 | 1213 |
| SAMMSON-224 | ENST00000641929.1 | 849 |
| SAMMSON-225 | ENST00000642004.1 | 1199 |
| SAMMSON-226 | ENST00000642089.1 | 1582 |
| SAMMSON-227 | ENST00000642114.1 | 1199 |

TABLE 2
Annotated SAMMSON transcripts in LNCipedia

| Transcript ID | Gene ID | Location (hg38) | strand | transcript size |
|---|---|---|---|---|
| SAMMSON:1 | SAMMSON | chr3:69999577-70013867 | + | 513 |
| SAMMSON:10 | SAMMSON | chr3:69999733-70105208 | + | 34263 |
| SAMMSON:11 | SAMMSON | chr3:69999733-70142525 | + | 3059 |
| SAMMSON:12 | SAMMSON | chr3:70065114-70070449 | + | 2457 |
| SAMMSON:13 | SAMMSON | chr3:70082434-70097988 | + | 2024 |
| SAMMSON:14 | SAMMSON | chr3:70140217-70140488 | + | 272 |
| SAMMSON:15 | SAMMSON | chr3:69999550-70072666 | + | 1657 |
| SAMMSON:16 | SAMMSON | chr3:69999556-70068710 | + | 1046 |
| SAMMSON:17 | SAMMSON | chr3:69999557-70030724 | + | 849 |
| SAMMSON:18 | SAMMSON | chr3:69999572-70172924 | + | 1162 |
| SAMMSON:19 | SAMMSON | chr3:69999572-70184197 | + | 1487 |
| SAMMSON:2 | SAMMSON | chr3:69999744-70015318 | + | 2044 |
| SAMMSON:20 | SAMMSON | chr3:69999572-70014782 | + | 1433 |
| SAMMSON:21 | SAMMSON | chr3:69999576-70127252 | + | 1582 |
| SAMMSON:22 | SAMMSON | chr3:69999577-70205153 | + | 1199 |
| SAMMSON:23 | SAMMSON | chr3:69999583-70184202 | + | 1377 |
| SAMMSON:24 | SAMMSON | chr3:69999583-70120708 | + | 1264 |
| SAMMSON:25 | SAMMSON | chr3:69999587-70014764 | + | 1521 |
| SAMMSON:26 | SAMMSON | chr3:69999594-70172924 | + | 1213 |
| SAMMSON:27 | SAMMSON | chr3:69999599-70172924 | + | 1207 |
| SAMMSON:28 | SAMMSON | chr3:69999599-70205148 | + | 1308 |
| SAMMSON:29 | SAMMSON | chr3:69999607-70072666 | + | 1847 |
| SAMMSON:3 | SAMMSON | chr3:69999744-70015298 | + | 2024 |
| SAMMSON:30 | SAMMSON | chr3:69999733-70075385 | + | 1110 |
| SAMMSON:31 | SAMMSON | chr3:69999733-70184202 | + | 1546 |
| SAMMSON:32 | SAMMSON | chr3:69999733-70014787 | + | 1645 |
| SAMMSON:33 | SAMMSON | chr3:69999734-70272334 | + | 1169 |
| SAMMSON:34 | SAMMSON | chr3:69999744-70015301 | + | 2027 |
| SAMMSON:35 | SAMMSON | chr3:69999748-70070336 | + | 2727 |
| SAMMSON:36 | SAMMSON | chr3:69999775-70184125 | + | 1355 |
| SAMMSON:37 | SAMMSON | chr3:69999784-70025206 | + | 868 |
| SAMMSON:38 | SAMMSON | chr3:69999788-70205924 | + | 2168 |
| SAMMSON:39 | SAMMSON | chr3:69999846-70389859 | + | 1199 |
| SAMMSON:4 | SAMMSON | chr3:69999744-70015300 | + | 2026 |
| SAMMSON:41 | SAMMSON | chr3:70283611-70425190 | + | 1100 |
| SAMMSON:42 | SAMMSON | chr3:70332740-70518064 | + | 1516 |
| SAMMSON:43 | SAMMSON | chr3:70167806-70172562 | + | 995 |
| SAMMSON:5 | SAMMSON | chr3:70012417-70015014 | + | 1649 |
| SAMMSON:6 | SAMMSON | chr3:69999733-70003043 | + | 3311 |
| SAMMSON:7 | SAMMSON | chr3:69999733-70462735 | + | 908 |
| SAMMSON:8 | SAMMSON | chr3:69999733-70015318 | + | 2055 |
| SAMMSON:9 | SAMMSON | chr3:69999733-70016525 | + | 3383 |

Note however that, for all sequences, variations in the non-coding exons have been reported in dbSNP, and these variations are envisaged as belonging to the respective transcript IDs. I.e., unless specifically mentioned otherwise, the term SAMMSON encompasses the different isoforms.

With “functional expression” of SAMMSON, it is meant the transcription and/or translation of functional gene product. In other words, the inhibitors of the disclosure target the SAMMSON gene or transcript directly by way of sequence complementarity. For non-protein coding genes like SAMMSON, “functional expression” can thus be deregulated on at least two levels. First, at the DNA level, e.g., by absence or disruption of the gene, or lack of transcription taking place (in both instances preventing synthesis of the relevant gene product). The lack of transcription can, e.g., be caused by epigenetic changes (e.g., DNA methylation) or by loss of function mutations. A “loss-of-function” or “LOF” mutation as used herein is a mutation that prevents, reduces or abolishes the function of a gene product as opposed to a gain-of-function mutation that confers enhanced or new activity on a protein. LOF can be caused by a wide range of mutation types, including, but not limited to, a deletion of the entire gene or part of the gene, splice site mutations, frame-shift mutations caused by small insertions and deletions, nonsense mutations, missense mutations replacing an essential amino acid and mutations preventing correct cellular localization of the product. Also included within this definition are mutations in promoters or regulatory regions of the SAMMSON gene if these interfere with gene function. A null mutation is an LOF mutation that completely abolishes the function of the gene product. A null mutation in one allele will typically reduce expression levels by 50%, but may have severe effects on the function of the gene product. Note that functional expression can also be deregulated because of a gain of function mutation: by conferring a new activity on the protein, the normal function of the protein is deregulated, and less functionally active protein is expressed. Vice versa, functional expression can be increased, e.g., through gene duplication or by lack of DNA methylation. Second, at the RNA level, e.g., by lack of efficient translation taking place—e.g., because of destabilization of the mRNA (e.g., by UTR variants) so that it is degraded before translation occurs from the transcript. Or by lack of efficient transcription, e.g., because a mutation introduces a new splicing variant. The term “status” as used in the application with regard to a particular protein, specifically tumor associated proteins (e.g., p53 status, BRAF status, NRAS status, MEK status, . . . ) refers to the mutational status and/or the expression of these particular proteins. Typically, the term is used in the sense “irrespective of” or “independent of” status, meaning that an effect is observed irrespective of expression levels of, or presence of mutations in, the particular protein.

“Long non-coding RNAs” (long ncRNAs, lncRNAs) as used herein are non-protein coding transcripts longer than 200 nucleotides. A particular class of lncRNA are long intergenic ncRNAs (lincRNAs), referring to long non-coding RNAs that are transcribed from non-coding DNA sequences between protein-coding genes.

The application shows specific expression of lncRNAs in uveal melanoma. Inhibition of such lncRNA can be used to selectively induce apoptosis in these cancer cells.

Accordingly, provided are inhibitors of functional expression of the SAMMSON gene. Such inhibitors can act at the DNA level, or at the RNA (i.e., gene product) level. As SAMMSON is a non-coding gene, there is no protein product for this gene. In other words, the latter inhibitors target the SAMMSON gene or transcript directly by way of sequence complementarity.

If inhibition is to be achieved at the DNA level, this may be done using gene therapy to knock-out or disrupt the target gene. As used herein, a “knock-out” can be a gene knockdown or the gene can be knocked out by a mutation such as, a point mutation, an insertion, a deletion, a frameshift, or a missense mutation by techniques known in the art, including, but not limited to, retroviral gene transfer. Another way in which genes can be knocked out is by the use of zinc finger nucleases. Zinc finger nucleases (ZFNs) are artificial restriction enzymes generated by fusing a zinc finger DNA-binding domain to a DNA-cleavage domain. Zinc finger domains can be engineered to target desired DNA sequences, which enable zinc-finger nucleases to target unique sequence within a complex genome. By taking advantage of endogenous DNA repair machinery, these reagents can be used to precisely alter the genomes of higher organisms. Other technologies for genome customization that can be used to knock out genes are meganucleases and TAL effector nucleases (TALENs, Cellectis bioresearch). A TALEN® is composed of a TALE DNA binding domain for sequence-specific recognition fused to the catalytic domain of an endonuclease that introduces double strand breaks (DSB). The DNA binding domain of a TALEN® is capable of targeting with high precision a large recognition site (for instance, 17 bp). Meganucleases are sequence-specific endonucleases, naturally occurring “DNA scissors,” originating from a variety of single-celled organisms such as bacteria, yeast, algae and some plant organelles. Meganucleases have long recognition sites of between 12 and 30 base pairs. The recognition site of natural meganucleases can be modified in order to target native genomic DNA sequences (such as endogenous genes).

Gene inactivation, i.e., inhibition of functional expression of the gene, may, for instance, also be achieved through the creation of transgenic organisms expressing antisense RNA, or by administering antisense RNA to the subject. An antisense construct can be delivered, for example, as an expression plasmid, which, when transcribed in the cell, produces RNA that is complementary to at least a unique portion of the cellular SAMMSON lncRNA. A more rapid method for the inhibition of gene expression is based on the use of shorter antisense oligomers consisting of DNA, or other synthetic structural types such as phosphorothiates, 2′-0-alkylribonucleotide chimeras, locked nucleic acid (LNA), 2′,4′-constrained ethyl nucleic acid (cET), 2′-O-methoxyethyl (2′-MOE), peptide nucleic acid (PNA), or morpholinos. With the exception of RNA oligomers, PNAs and morpholinos, all other antisense oligomers act in eukaryotic cells through the mechanism of RNase H-mediated target cleavage. PNAs and morpholinos bind complementary DNA and RNA targets with high affinity and specificity, and thus act through a simple steric blockade of the RNA translational machinery, and appear to be completely resistant to nuclease attack. An “antisense oligomer” refers to an antisense molecule or anti-gene agent that comprises an oligomer of at least about 10 nucleotides in length. In embodiments an antisense oligomer comprises at least 15, 18 20, 25, 30, 35, 40, or 50 nucleotides. Antisense approaches involve the design of oligonucleotides (either DNA or RNA, or derivatives thereof) that are complementary to an RNA encoded by polynucleotide sequences of SAMMSON. Antisense RNA may be introduced into a cell to inhibit translation of a complementary mRNA by base pairing to it and physically obstructing the translation machinery. This effect is therefore stoichiometric. Absolute complementarity, although preferred, is not required. A sequence “complementary” to a portion of an RNA, as referred to herein, means a sequence having sufficient complementarity to be able to hybridize with the RNA, forming a stable duplex; in the case of double stranded antisense polynucleotide sequences, a single strand of the duplex DNA may thus be tested, or triplex formation may be assayed. The ability to hybridize will depend on both the degree of complementarity and the length of the antisense polynucleotide sequence. Generally, the longer the hybridizing polynucleotide sequence, the more base mismatches with an RNA it may contain and still form a stable duplex (or triplex, as the case may be). One skilled in the art can ascertain a tolerable degree of mismatch by use of standard procedures to determine the melting point of the hybridized complex. Antisense oligomers should be at least 10 nucleotides in length, and are preferably oligomers ranging from 15 to about 50 nucleotides in length. In certain embodiments, the oligomer is at least 15 nucleotides, at least 18 nucleotides, at least 20 nucleotides, at least 25 nucleotides, at least 30 nucleotides, at least 35 nucleotides, at least 40 nucleotides, or at least 50 nucleotides in length. A related method uses ribozymes instead of antisense RNA. Ribozymes are catalytic RNA molecules with enzyme-like cleavage properties that can be designed to target specific RNA sequences. Successful target gene inactivation, including temporally and tissue-specific gene inactivation, using ribozymes has been reported in mouse, zebrafish and fruit flies. RNA interference (RNAi) is a form of post-transcriptional gene silencing. The phenomenon of RNA interference was first observed and described in Caenorhabditis elegans where exogenous double stranded RNA (dsRNA) was shown to specifically and potently disrupt the activity of genes containing homologous sequences through a mechanism that induces rapid degradation of the target RNA.

Several reports describe the same catalytic phenomenon in other organisms, including experiments demonstrating spatial and/or temporal control of gene inactivation, including plant (Arabidopsis thaliana), protozoan (Trypanosoma bruceii), invertebrate (Drosophila melanogaster), and vertebrate species (Danio rerio and Xenopus laevis). The mediators of sequence-specific messenger RNA degradation are small interfering RNAs (siRNAs) generated by ribonuclease III cleavage from longer dsRNAs. Generally, the length of siRNAs is between 20-25 nucleotides (Elbashir et al. (2001) Nature 411, 494 498). The siRNA typically comprise a sense RNA strand and a complementary antisense RNA strand annealed together by standard Watson Crick base pairing interactions (hereinafter “base paired”). The sense strand comprises a nucleic acid sequence that is identical to a target sequence contained within the target mRNA. The sense and antisense strands of the present siRNA can comprise two complementary, single stranded RNA molecules or can comprise a single molecule in which two complementary portions are base paired and are covalently linked by a single stranded “hairpin” area (often referred to as shRNA). The term “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered.

The siRNAs of the disclosure can comprise partially purified RNA, substantially pure RNA, synthetic RNA, or recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siRNA or to one or more internal nucleotides of the siRNA, including modifications that make the siRNA resistant to nuclease digestion. One or both strands of the siRNA of the disclosure can also comprise a 3′ overhang. A “3′ overhang” refers to at least one unpaired nucleotide extending from the 3′ end of an RNA strand. Thus, in one embodiment, the siRNA of the disclosure comprises at least one 3′ overhang of from one to about six nucleotides (which includes ribonucleotides or deoxynucleotides) in length, preferably from one to about five nucleotides in length, more preferably from one to about four nucleotides in length, and particularly preferably from about one to about four nucleotides in length.

In the embodiment in which both strands of the siRNA molecule comprise a 3′ overhang, the length of the overhangs can be the same or different for each strand. In a most preferred embodiment, the 3′ overhang is present on both strands of the siRNA, and is two nucleotides in length. In order to enhance the stability of the present siRNAs, the 3′ overhangs can also be stabilized against degradation. In one embodiment, the overhangs are stabilized by including purine nucleotides, such as adenosine or guanosine nucleotides.

Alternatively, substitution of pyrimidine nucleotides by modified analogues, e.g., substitution of uridine nucleotides in the 3′ overhangs with 2′ deoxythymidine, is tolerated and does not affect the efficiency of RNAi degradation. In particular, the absence of a 2′ hydroxyl in the 2′ deoxythymidine significantly enhances the nuclease resistance of the 3′ overhang in tissue culture medium. The siRNAs of the disclosure can be targeted to any stretch of approximately 19 to 25 contiguous nucleotides in any of the target SAMMSON RNA sequences (the “target sequence”), of which examples are given in the application. Techniques for selecting target sequences for siRNA are well known in the art. Thus, the sense strand of the present siRNA comprises a nucleotide sequence identical to any contiguous stretch of about 19 to about 25 nucleotides in the target mRNA. The siRNAs of the disclosure can be obtained using a number of techniques known to those of skill in the art. For example, the siRNAs can be chemically synthesized or recombinantly produced using methods known in the art. Preferably, the siRNA of the disclosure are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK). Alternatively, siRNA can also be expressed from recombinant circular or linear DNA plasmids using any suitable promoter. Suitable promoters for expressing siRNA of the disclosure from a plasmid include, for example, the U6 or H1 RNA pol III promoter sequences and the cytomegalovirus promoter. Selection of other suitable promoters is within the skill in the art. The recombinant plasmids of the disclosure can also comprise inducible or regulatable promoters for expression of the siRNA in a particular tissue or in a particular intracellular environment. The siRNA expressed from recombinant plasmids can either be isolated from cultured cell expression systems by standard techniques, or can be expressed intracellularly, e.g., in breast tissue or in neurons. The siRNAs of the disclosure can also be expressed intracellularly from recombinant viral vectors. The recombinant viral vectors comprise sequences encoding the siRNAs of the disclosure and any suitable promoter for expressing the siRNA sequences. Suitable promoters include, for example, the U6 or H1 RNA pol III promoter sequences and the cytomegalovirus promoter. Selection of other suitable promoters is within the skill in the art. The recombinant viral vectors of the disclosure can also comprise inducible or regulatable promoters for expression of the siRNA in the tissue where the tumor is localized. As used herein, an “effective amount” of the siRNA is an amount sufficient to cause RNAi mediated degradation of the target mRNA, or an amount sufficient to inhibit the progression of metastasis in a subject. RNAi mediated degradation of the target mRNA can be detected by measuring levels of the target mRNA or protein in the cells of a subject, using standard techniques for isolating and quantifying mRNA or protein as described above. One skilled in the art can readily determine an effective amount of the siRNA of the disclosure to be administered to a given subject, by taking into account factors such as the size and weight of the subject; the extent of the disease penetration; the age, health and sex of the subject; the route of administration; and whether the administration is regional or systemic. Generally, an effective amount of the siRNA of the disclosure comprises an intracellular concentration of from about 1 nanomolar (nM) to about 100 nM, preferably from about 2 nM to about 50 nM, more preferably from about 2.5 nM to about 10 nM. It is contemplated that greater or lesser amounts of siRNA can be administered. Recently it has been shown that morpholino antisense oligonucleotides in zebrafish and frogs overcome the limitations of RNase H-competent antisense oligonucleotides, which include numerous non-specific effects due to the non-target-specific cleavage of other mRNA molecules caused by the low stringency requirements of RNase H. Morpholino oligomers therefore represent an important new class of antisense molecule. Oligomers of the disclosure may be synthesized by standard methods known in the art. As examples, phosphorothioate oligomers may be synthesized by the method of Stein et al. (1988) Nucleic Acids Res. 16, 3209 3021), methylphosphonate oligomers can be prepared by use of controlled pore glass polymer supports (Sarin et al. (1988) Proc. Natl. Acad. Sci. USA. 85, 7448-7451). Morpholino oligomers may be synthesized by the method of Summerton and Weller U.S. Pat. Nos. 5,217,866 and 5,185,444. Another particularly form of antisense RNA strategy are gapmers. A gapmer is a chimeric antisense oligonucleotide that contains a central block of deoxynucleotide monomers sufficiently long to induce RNase H cleavage. The central block of a gapmer is flanked by blocks of 2′-O modified ribonucleotides or other artificially modified ribonucleotide monomers such as bridged nucleic acids (BNAs) that protect the internal block from nuclease degradation. Gapmers have been used to obtain RNase-H mediated cleavage of target RNAs, while reducing the number of phosphorothioate linkages. Phosphorothioates possess increased resistance to nucleases compared to unmodified DNA. However, they have several disadvantages. These include low binding capacity to complementary nucleic acids and non-specific binding to proteins that cause toxic side-effects limiting their applications. The occurrence of toxic side-effects together with non-specific binding causing off-target effects has stimulated the design of new artificial nucleic acids for the development of modified oligonucleotides that provide efficient and specific antisense activity in vivo without exhibiting toxic side-effects. By recruiting RNase H, gapmers selectively cleave the targeted oligonucleotide strand. The cleavage of this strand initiates an antisense effect. This approach has proven to be a powerful method in the inhibition of gene functions and is emerging as a popular approach for antisense therapeutics. Gapmers are offered commercially, e.g., LNA longRNA GapmeRs by Qiagen, or MOE gapmers and cET gapmers by IONIS pharmaceuticals. MOE gapmers or “2′MOE gapmers” are antisense phosphorothioate oligonucleotides of 15-30 nucleotides wherein all of the backbone linkages are modified by adding a sulfur at the non-bridging oxygen (phosphorothioate) and a stretch of at least 10 consecutive nucleotides remain unmodified (deoxy sugars) and the remaining nucleotides contain an O′-methyl O′-ethyl substitution at the 2′ position (MOE).

The FDA-approved compounds of the disclosure relates to the following compounds:

| | | | Catalog | Chemical | |
|---|---|---|---|---|---|
| Compound | IUPAC name | Provider | number | formula | Status |
| Amsacrine | N-{4-[(acridin-9-yl)amino]-3- | MedChemExpress | HY- | C₂₁H₁₉N₃O₃S | Approved |
| (m-AMSA) | methoxyphenyl}methanesulfonamide | | 13551 | | (acute myeloid |
| | | | | | leukemia) |
| Entinostat | (pyridin-3-yl)methyl N-({4-[(2- | Selleckchem | S1053 | C21H20N4O3 | phase |
| (MS-275) | aminophenyl)carbamoyl]phenyl}methyl)carbamate | | | | II(-III) |
| INH-6 | N-[4-(2,4,6-trimethylphenyl)-2- | Selleckchem | S7494 | C19H18N2OS | |
| | thiazolyl]-benzamide | | | | |
| Mifepristone | (1S,3aS,3bS,10R,11 aS)-10-[4- | Selleckchem | S2606 | C29H35NO2 | Approved |
| (RU486) | (dimethylamino)phenyl]-1-hydroxy- | | | | (termination of |
| | 11a-methyl-1-(prop-1-yn-1-yl)- | | | | intrauterine |
| | 1H,2H,3H,3aH,3bH,4H,5H,7H,8H,9H, | | | | pregnancy) |
| | 10H,11H,11aH- | | | | |
| | cyclopenta[a]phenanthren-7-one | | | | |
| CI-994 | N-(2-aminophenyl)-4- | Selleckchem | S2818 | C15H15N3O2 | phase II-III |
| (Tacedinaline) | acetamidobenzamide | | | | |
| Clioquinol | 5-chloro-7-iodoquinolin-8-ol | Selleckchem | S4601 | C9H5CIINO | Approved |
| | | | | | (topical antifungal |
| | | | | | treatment), |
| | | | | | withdrawn due to |
| | | | | | neurotoxicity |
| GDC-0349 | 3-ethyl-1-(4-{4-[(3S)-3- | Selleckchem | S8040 | C24H32N6O3 | Phase I |
| (RG-7603) | methylmorpholin-4-yl]-7-(oxetan-3-yl)- | | | | |
| | 5H,6H,7H,8H-pyrido[3,4-d]pyrimidin- | | | | |
| | 2-yl}phenyl)urea | | | | |

### EXAMPLES

### Materials and Methods

### Cell Culture

The human uveal melanoma cell lines 92.1 and OMM1 were obtained from the Leiden University Medical Center. The cell lines were cultivated in Dulbecco's modified Eagle's medium (DMEM, Gibco)/F12—Roswell Park Memorial Institute (RPMI, Gibco) 1640 (1:1) medium and supplemented with 10% fetal bovine serum (FBS), 2 mM L-glutamine (Gibco) and 100 IU/ml Penicillin/Streptomycin (Gibco). All cell lines were incubated in a humidified atmosphere containing 5% CO₂ at 37° C. For the execution of the experiments cells were incubated in L-glutamine and Penicillin/Streptomycin free media. Short tandem repeat (STR) genotyping was used to validate cell line authenticity and Mycoplasma testing was done on a monthly basis.

### Compounds and Antisense Oligonucleotides (ASOs)

The 2911 compounds used in the screening were obtained from the centre for drug design and discovery (CD3, KU Leuven). For the further validation experiments, the compounds were purchased separately. Amsacrine (4-[9-acridinylamino]-N-[methanesulfonyl]-m-anisidine hydrochloride) was purchased from MedChemExpress. Pimozide (3-[1-[4,4-bis(4-fluorophenyl)butyl]piperidin-4-yl]-1H-benzimidazol-2-one) was purchased from Sigma Aldrich. CI-994 (N-(2-aminophenyl)-4-acetamidobenzamide), Clioquinol (5-chloro-7-iodoquinolin-8-ol), Ebastine (4-(4-benzhydryloxypiperidin-1-yl)-1-(4-tert-butylphenyl)butan-1-one), Entinostat ((pyridin-3-yl)methyl N-({4-[(2-aminophenyl)carbamoyl]phenyl}methyl)carbamate), Fenofibric acid (2-[4-(4-chlorobenzoyl)phenoxy]-2-methylpropanoic acid), Fluphenazine dihydrochloride (2-[4-[3-[2-(trifluoromethyl)phenothiazin-10-yl]propyl]piperazin-1-yl]ethanol; dihydrochloride), GDC-0349 (3-ethyl-1-(4-{4-[(3S)-3-methylmorpholin-4-yl]-7-(oxetan-3-yl)-5H,6H,7H, 8H-pyrido[3,4-d]pyrimidin-2-yl}phenyl)urea), INH-6 (N-[4-(2,4,6-trimethylphenyl)-2-thiazolyl]-benzamide), LDN-193189 2HCl (4-[6-(4-piperazin-1-ylphenyl)pyrazolo[1,5-a]pyrimidin-3-yl]quinoline; hydrochloride), Mifepristone ((1S,3aS,3bS,10R,11aS)-10-[4-(dimethylamino)phenyl]-1-hydroxy-11a-methyl-1-(prop-1-yn-1-yl)-1H,2H,3H,3aH,3bH,4H,5H,7H, 8H,9H,10H,11H,11aH-cyclopenta[a]phenanthren-7-one), Oltiprax (4-methyl-5-pyrazin-2-yldithiole-3-thione), Oxolinic acid (5-ethyl-8-oxo-[1,3]dioxolo[4,5-g]quinoline-7-carboxylic acid), Perphenazine (2-[4-[3-(2-chlorophenothiazin-10-yl)propyl]piperazin-1-yl]ethanol), Propylthiouracil (6-propyl-2-sulfanylidene-1H-pyrimidin-4-one), Ranitidine hydrochloride ((E)-1-N′-[2-[[5-[(dimethylamino)methyl]furan-2-yl]methyl sulfanyl]ethyl]-1-N-methyl-2-nitroethene-1,1-diamine; hydrochloride) and Riboflavin (7,8-dimethyl-10-[(2S,3S,4R)-2,3,4,5-tetrahydroxypentyl]benzo[g]pteridine-2,4-dione) were purchased from Selleckchem.

The LNA GapmeR oligonucleotides specifically targeting SAMMSON and the LNA non-targeting control (NTC) GapmeR were purchased from Qiagen.

Sequence LNA ASO 3:
(SEQ ID NO: 1)
GTGTGAACTTGGCT
Sequence LNA ASO 11:
(SEQ ID NO: 2)
TTTGAGAGTTGGAGGA
Sequence LNA NTC:
(SEQ ID NO: 3)
TCATACTATATGACAG

### ASO-Compound Screening

For the ASO-compound screening, uveal melanoma cell line 92.1 was seeded in opaque 384 well plates (Greiner Bio-one, cat #788010) at a density of 1250 cells/well. After overnight incubation, the cells were transfected using lipofectamine 2000 (Life Technologies) with 35 nM ASO 3 or NTC using a Multidrop Combi reagent Dispenser (Thermo Fisher Scientific). From each compound plate, compounds were added using the Janus Mini MDT workstation (PerkinElmer) to an ASO 3 transfected plate and a NTC transfected plate, to a final compound concentration of 10 μM and final DMSO concentration of 0.1%. Cell viability was examined 48 h after treatment using a CellTiter-Glo assay (Promega). Before initiating the assay, the culture plates and reconstituted assay buffer were placed at room temperature for 15 minutes. Next, 20 μl of cellTiter-Glo reagent was added per well and plates were incubated for 15 minutes at room temperature to induce cell lysis. The luminescence signal was measured with an Envision plate reader (PerkinElmer). The confirmation experiment was performed in the same way as described above, but 4 replicates/condition were included.

For the validation experiments, uveal melanoma cell lines 92.1 and OMM1 were seeded in 96 well plates (Corning costar 3596) at a density of 5000 cells/well and were allowed to settle overnight. Subsequently, the cells were transfected using lipofectamine 2000 (Life Technologies) with 50 or 100 nM LNA ASO 3 or LNA NTC and treated with various concentrations of the compounds and with a final DMSO concentration of maximum 0.1%. The compounds were added to the wells using the HP D300e Digital dispenser (Tecan). Control cells were transfected with LNA NTC ASO and treated with DMSO.

Cell viability was also examined using a CellTiter-Glo assay (Promega). Before initiating the assay, the culture plates and reconstituted assay buffer were placed at room temperature for 30 minutes. Next, the culture medium was replaced by 200 μl fresh culture medium—- assay buffer (1:1) mix. To induce complete cell lysis, the plates were shaken for 10 min. 100 μl from each well was subsequently transferred to an opaque 96-well plate (Nunc), which was measured with a GloMax 96 Microplate Luminometer (Promega). The CellTiter-Glo assay was performed on various predefined timepoints.

For real-time analysis, IncuCyte Zoom system and IncuCyte S3 system (Essen BioScience) were used. Cells were seeded and treated as described above. After treatment, the culture plate was incubated in an IncuCyte Zoom system or IncuCyte S3 system at 37° C. in a humidified 5% CO₂ incubator. Phase contrast whole well images were captured every 3 h. The IncuCyte ZOOM and IncuCyte S3 software (Essen BioScience) were utilized in real-time to measure % confluence, as a proxy for proliferation.

### Monitoring of Synergistic ASO-Compound Combinations

Bliss independence (BI) scores were calculated to determine ASO-compound interactions. The predictive performance (BI score) is then compared to the observed drug interaction data measured by the Excess over Bliss. Excess over Bliss scores>0 indicate drug synergism, Excess over Bliss scores=0 indicate additive effects and Excess over Bliss scores<0 indicate drug antagonism.

### Western Blot Analysis

Cells were lysed in RIPA lysis buffer (5 mg/ml sodium deoxycholate, 150 mM NaCl, 50 mM Tris-HCl pH 7.5, 0.1% SDS solution, 1% NP-40) supplemented with protease and/or phosphatase inhibitors Protein concentrations were determined with the BCA protein assay (Bio-Rad). In total, 35 μg of protein lysate was loaded onto an SDS-PAGE gel (10% Pre-cast, Bio-Rad), run at 100 V for 1 h and subsequently blotted onto a nitrocellulose membrane. Antibodies against phospho-S6 Ribosomal Protein (Ser235/236) (#2211, 1:1000 dilution), phospho-4E-BP(Thr37/46) (#2855, 1:1000 dilution), S6 Ribosomal Protein (#2317, 1:1000 dilution) and 4E-BP1 (#9644, 1:1000 dilution) were purchased from Cell Signaling Technology. HRP-labeled anti-rabbit (7074 S, Cell Signaling, 1:10 000 dilution) and anti-mouse (7076P2, Cell Signaling, 1:10 000 dilution) antibodies were used as secondary antibodies. Anti-Vinculin antibody (V9131, Sigma-Aldrich, 1:10 000 dilution) was used as loading control. The antibodies were diluted in BSA/TBST (5% BSA in TBS with 0.1% Tween20) and antibody binding with membrane was evaluated using the SuperSignal West Dura Extended Duration Substrate (ThermoFisher Scientific) or SuperSignal West Femto Maximum Sensitivity Substrate (ThermoFisher Scientific). Imaging was performed by means of the Amersham Imager 680 (GE Healthcare). Image J was used for the quantification of the blots.

### SUNSET

Cells were seeded in T75 culture flasks (Cellstar) at a density of 1 170 000 cells/T75 flask 24 h prior to transfection. The cells were transfected with 50 nM LNA ASO 3 or NTC using lipofectamine 2000, followed by GDC-0349 (0.625 μM) or DMSO treatment. 24 h later, cells were washed in 1× phosphate buffer saline (PBS) and subsequently incubated with puromycin containing media (InvivoGen, 10 μg/ml) for 10 min. Puromycin incorporation is a proxy for the mRNA translation rate in vitro and was measured by Western blotting using an anti-puromycin antibody (MABE343, clone 12D10, Merck Millipore, 1:10 000). The antibody was diluted in Milk/TB ST (5% non-fat dry milk in TBS with 0.1% Tween20). Imaging was performed by means of the Amersham Imager 680 (GE Healthcare). An equal protein loading was verified using a ponceau S staining (Sigma Aldrich). Image J was used for the quantification of the blots.

### Differential Gene Expression Analysis by RNA Sequencing

RNA sequencing was performed on quadruplicates of NTC (50 nM), ASO 3 (50 nM), GDC-0349 (0.625 μM) and ASO 3 (50 nM)+GDC-0349 (0.625 μM) treated 92.1 and OMM1 cells. Libraries for RNA sequencing were prepared using the Quant Seq 3′ end library prep (Illumina) and quantified on a Qubit 2.0 Fluorometer prior to single-end sequencing with 75 bp read length on a NextSeq 500 sequencer (Illumina). Reads were mapped to the human genome (hg38) and gene expression was quantified using HTSeq. Differentialy expressed genes were identified using DESeq2.

### Seahorse XF Cell Mito Stress Test

Cells were seeded in T75 culture flasks (Cellstar) at a density of 1 170 000 cells/T75 flask 24 h prior to transfection. The cells were transfected with 50 nM ASO 3 or NTC using lipofectamine 2000, followed by GDC-0349 (0.625 μM) treatment. Four hours later, 25 000 cells were transferred to Seahorse XFp Cell Culture Miniplates (Agilent) and were allowed to settle overnight. Subsequently, oxygen consumption rates were measured in triplicates for each condition using the Seahorse XFp device (Agilent) according to the standard mito stress test procedures in seahorse assay medium supplemented with 14.3 mM glucose, 1 mM pyruvate and 2 mM glutamine (Sigma), and cells were sequentially challenged with 1 μM oligomycin, 1 μM carbonyl cyanide 4-(trifluoromethoxy)-phenylhydrazone (FCCP) and 0.5 μM of a rotenone antimycin A mix (Agilent). Following the assay, protein concentrations were calculated based upon absorbance reading at 280 nm (Biodrop, Isogen Lifescience) for normalization of the results. Spare respiratory capacity was calculated as the difference between maximal and the basal oxygen consumption rate. Relative oxygen consumption rates are relative compared to NTC values.

### Real-Time Quantitative PCR (RT-qPCR)

Total RNA was extracted using the miRNeasy kit (Qiagen) according to the manufacturer's instructions, including on-column DNase treatment. The Nanodrop (ThermoFisher Scientific) was used to determine RNA concentrations and cDNA synthesis was performed using the iScript Advanced cDNA synthesis kit (Bio-Rad) using a mix containing 200 ng of RNA, 4 μl of 5× iScript advanced reaction buffer and 1 μl of iScript advanced reverse transcriptase. The qPCR mix contains 2 μl of cDNA (5 ng), 2.5 μl SsoAdvanced Universal SYBR Green Supermix (Bio-Rad), 0.25 μl forward (5 μM, IDT) and reverse primer (5 μM, IDT) and was analyzed on a LC-480 device (Roche). Expression levels were normalized using expression data of at least 2 stable reference genes out of 4 tested reference genes (SDHA^{9,10}, HPRT1^{11,12}, UBC¹³ and TBP^{14,15}). Analysis was performed using the qbase+software (https://www.qbaseplus.com).

The olionucleotide primers used for qPCR were as follows:

SAMMSON
Fw:
(SEQ ID NO: 4)
CCTCTAGATGTGTAAGGGTAGT,
Rv:
(SEQ ID NO: 5)
TTGAGTTGCATAGTTGAGGAA
SDHA
Fw:
(SEQ ID NO: 6)
TGGGAACAAGAGGGCATCTG,
Rv:
(SEQ ID NO: 7)
CCACCACTGCATCAAATTCATG
HPRT1
Fw:
(SEQ ID NO: 8)
TGACACTGGCAAAACAATGCA,
Rv:
(SEQ ID NO: 9)
GGTCCTTTTCACCAGCAAGCT
UBC
Fw:
(SEQ ID NO: 10)
ATTTGGGTCGCGGTTCTTG,
Rv:
(SEQ ID NO: 11)
TGCCTTGACATTCTCGATGGT
TBP
Fw:
(SEQ ID NO: 12)
CACGAACCACGGCACTGATT,
Rv:
(SEQ ID NO: 13)
TTTTCTTGCTGCCAGTCTGGAC

### Statistical Analysis

Statistical analyses and data visualizations were performed with Graphpad Prism Version 8.0.2 (GraphPad Software, San Diego, Calif. USA).

### Results

### Up-Regulated SAMMSON Expression in Uveal Melanoma Tumors

PAN cancer RNA sequencing data from more than 10 000 tumor samples representing 32 cancer types (The Cancer Genome Atlas, TCGA) showed the highest and most consistent expression of SAMMSON in skin melanoma (SKCM, >90%). In addition, in more than 80% of all uveal melanoma tumors a consistent SAMMSON expression could be detected, although to lower levels compared to skin melanoma. As barely any expression of SAMMSON can be found in other tumor types, normal melanocytes and other normal tissues, an important function of SAMMSON for tumor cell survival is presumed. Despite the importance of SAMMSON expression for tumor cell survival, no link between the SAMMSON expression level and vital status of the UVM patients, clinical stage of tumor progression, tumor localization site (choroid, ciliary body or iris) or the level of metastasis could be observed. Tumorigenesis and progression of cancer are in general preceded by the occurrence of genetic changes in normal cells¹⁶. The recurrent aberrations in UM concern loss of 1 p, monosomy of chromosome 3, loss of 6 q and 8 p and gain of 6 p and 8 q. Poor prognosis and high risk of metastatic disease are often accompanied with loss of chromosome 3⁴. Strikingly, loss of one copy of chromosome 3 (location of SAMMSON) does not result in a reduction in SAMMSON expression, which hints toward the existence of a compensation mechanism.

Differential expression analysis revealed differential expression of 656 genes between monosomy 3 and disomy 3 UM tumors. SAMMSON is one of the 75 genes showing a maintained or up-regulated expression upon chromosome 3 loss.

Elevated SAMMSON expression is associated with UM cell proliferation Since SAMMSON expression is lower in UM tumors compared to SKCM tumors, the expression level of SAMMSON in multiple UVM cell lines by qRT-PCR was investigated. Comparing SAMMSON expression to SKCM cell line SK-MEL28, a sufficiently high expression level could be detected in multiple uveal melanoma cell lines originating from primary tumors (92.1, MEL270 and MP65) and metastatic tumors (OMM2.3, OMM1 and MM28). To evaluate the importance of SAMMSON in UM, the effects of SAMMSON knockdown on UM cell lines was studied using multiple independent SAMMSON-targeting antisense oligonucleotides (ASOs). Transfection of two independent LNA modified ASOs results in a dose dependent decrease of SAMMSON expression and consequentially turned out in a strong dose dependent reduction in cell viability, accompanied by induction of apoptosis in multiple UM cell lines. These results were irrespective to the mutational status of the UM cell lines as cell lines harboring GNAQ, GNA11 and BAP1 mutations were included. Comparable to SKCM¹⁷, LNA ASO 3 shows, in most cell lines, a more efficient knockdown and phenotypic effect compared to LNA ASO 11.

### SAMMSON Promotes Tumor Growth In Vivo

Since SAMMSON expression is not conserved across species, an UM patient derived xenografts (PDX) model (MP46) was used to investigate the effect on tumor growth in vivo upon SAMMSON knock down. LNA ASO 3 and LNA non targeting control (NTC) were subcutaneous injected at a dose of 10 mg/kg three times in the first week and twice per week in the second and third week. Upon SAMMSON knock down, a significant tumor growth inhibition could be observed compared to the NTC treated PDX mice. Notably, no weight loss could be observed after LNA ASO 3 administration, which indicates that the SAMMSON targeting ASO is not resulting in any toxicity effects.

### Unbiased Combination Screening Revealed Multiple Potential Compound-ASO Combinations for Treating UM

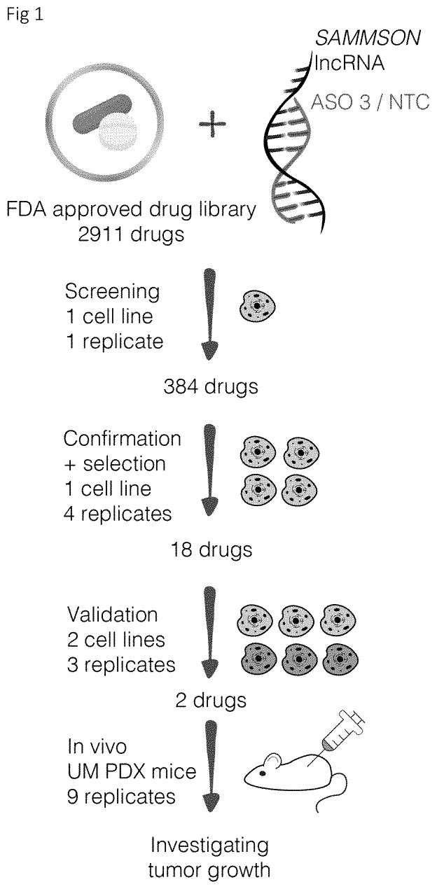

Tumor growth, survival, invasion and metastasis are dependent on more than one signaling pathway. These complex signaling networks, where oncogenes and oncogenic proteins interact through crosstalk and feedback loops, makes it difficult to obtain sustained targeting effects and avoid the development of drug resistance with single drugs. Monotherapy is still a common treatment modality, but the cancer complexity is pointing out the necessity of combination therapy approaches^{18,19}. In that perspective, an unbiased screening was performed to identify compounds resulting in an additional or synergistic effect when combined with the SAMMSON inhibiting ASO 3. An overview of the screening procedure can be found in FIG. 1. The compounds used in the screening were from an FDA approved compound library consisting of 2911 compounds, which are all in at least phase I clinical trial.

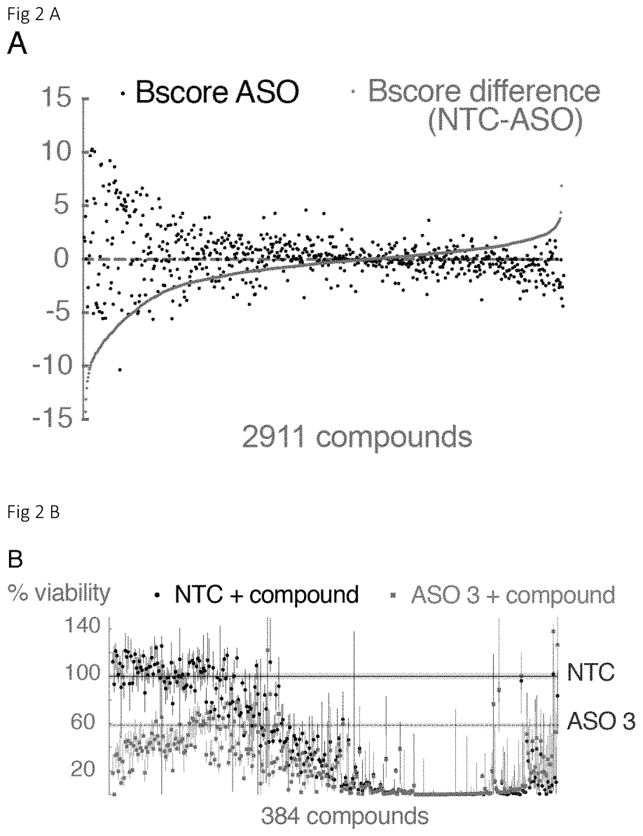

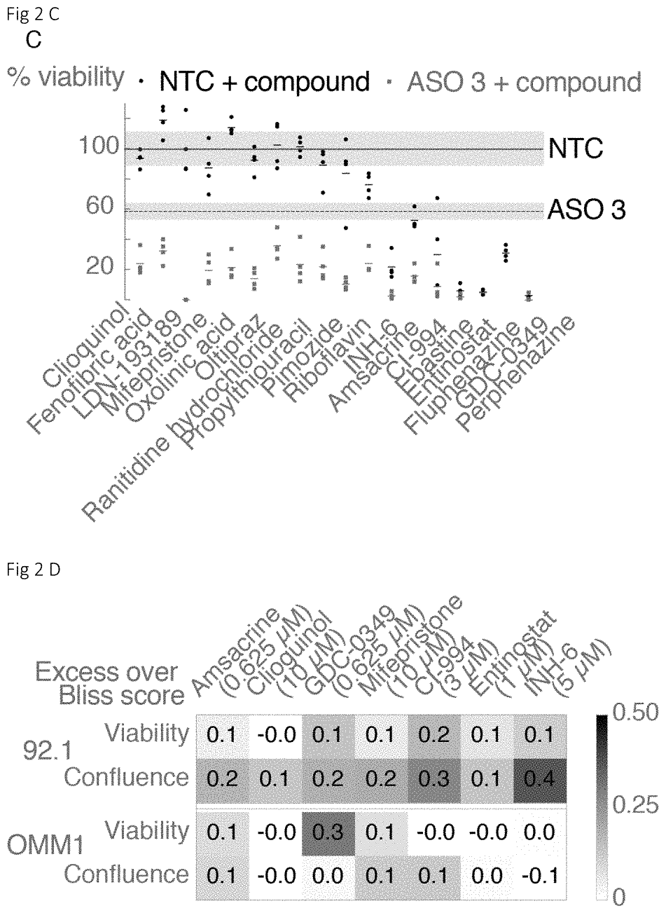

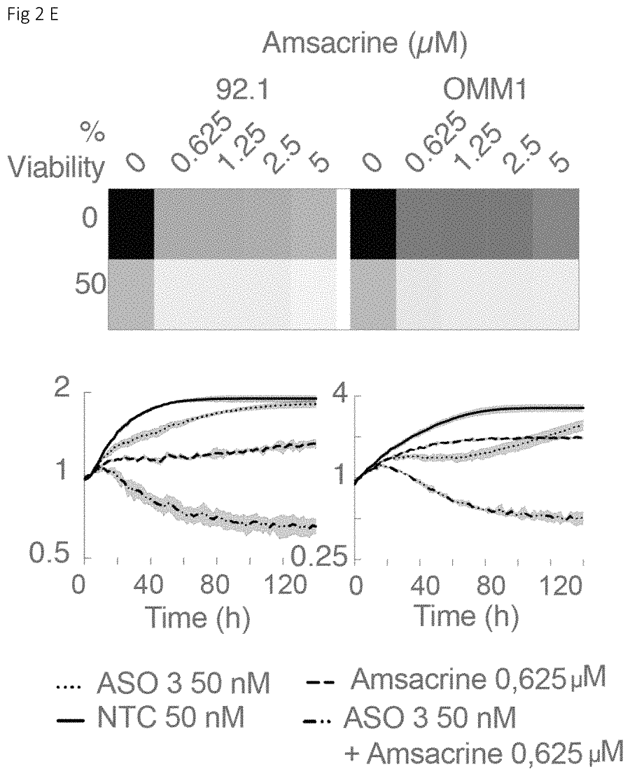

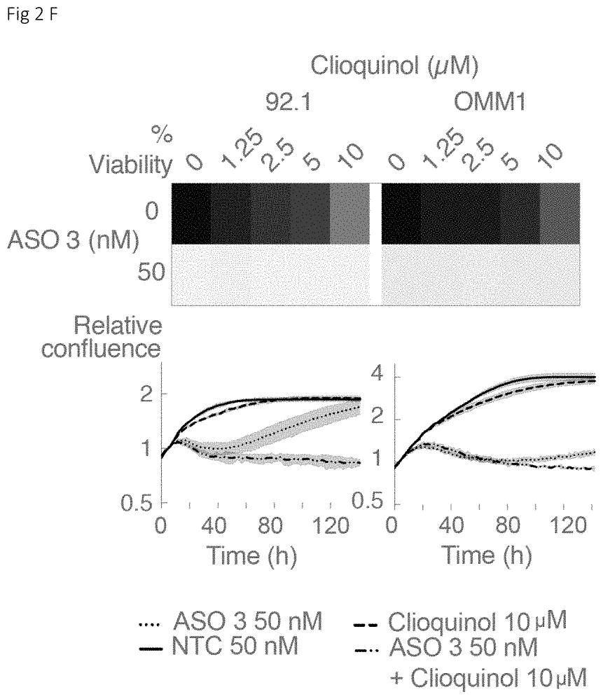

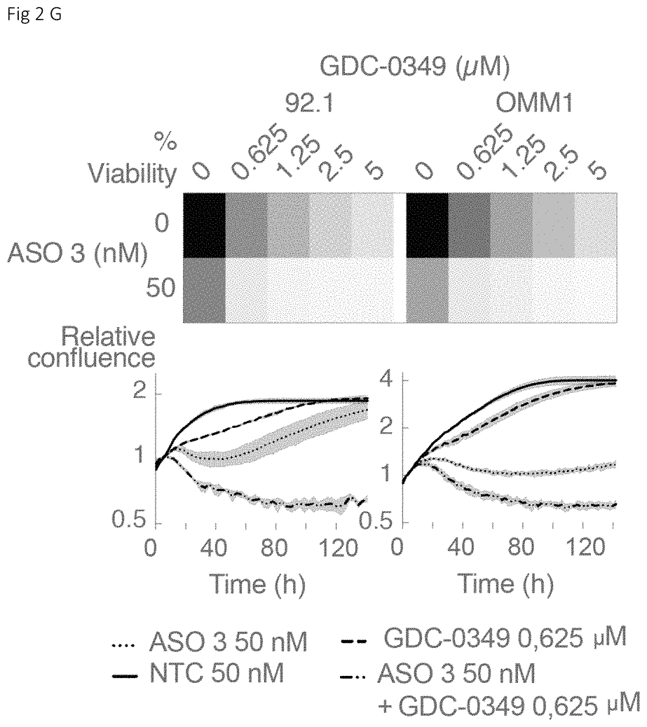

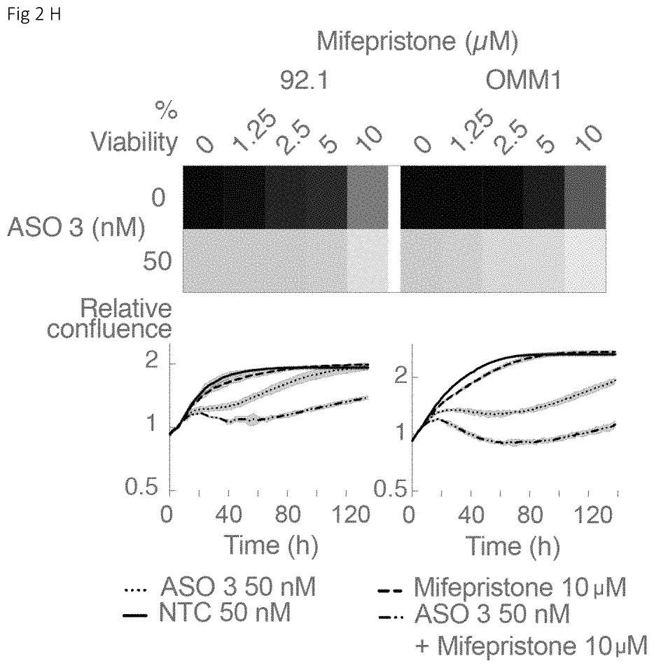

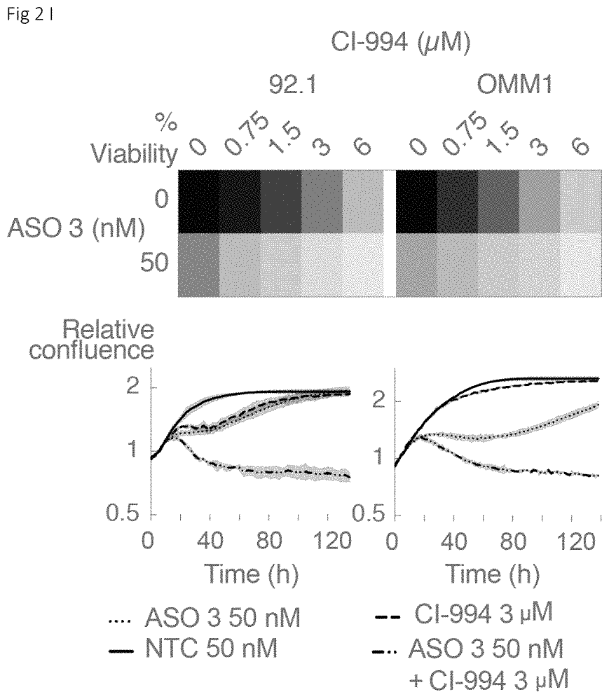

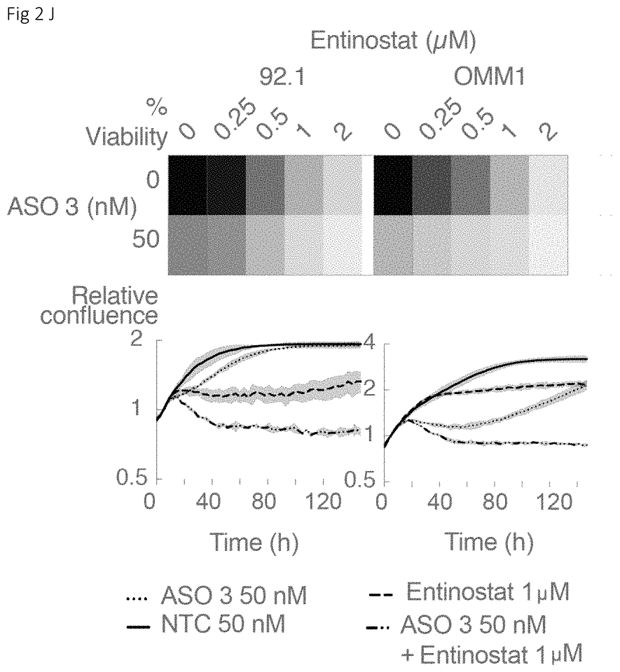

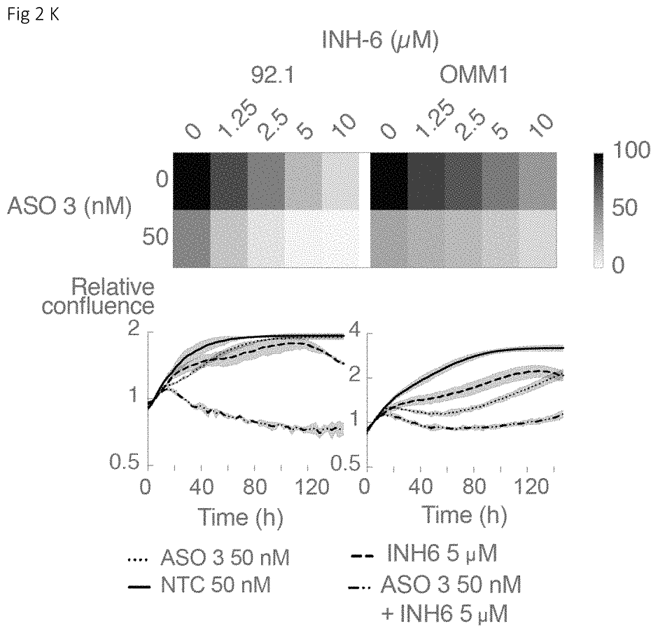

In the initial screening, performed on the UM cell line 92.1, a fixed concentration of ASO 3/NTC (35 nM) and compound (10 μM) was used to examine the viability effect in combination compared to ASO 3 and compound monotherapy. All combinations were run on multiple plates and to avoid plate effects, intraplate normalization via two-way median polish and interplate normalization via Bscore calculation were conducted. Bscores represent the effect of a compound on the viability compared to all samples in the same condition²⁰. To compare the relative viability for each compound in the NTC and the ASO 3 condition, the Bscores of the compounds in the ASO 3 condition were subtracted from the B scores in the NTC condition (Bscore difference (NTC−ASO)). 384 compounds were selected for a confirmation screen based on compounds resulting in negative Bscores (lower relative viability) and positive B score differences (lower relative viability in the ASO condition compared to the NTC condition) (FIG. 2A). The confirmation screen, conducted in quadruplicate, resulted in the selection of 18 compounds for further in vitro validation (FIGS. 2B-2C). Part of these compounds are not or only slightly affecting cell viability on their own, but when combining with the SAMMSON inhibiting ASO 3, a decrease in cell viability that goes beyond the ASO 3 effect could be observed. Based on fold change differences, other compounds were also selected that do show a viability effect, but in combination with ASO 3 are resulting in an additional/synergistic effect. Concentration- and time-dependent cytotoxic effects on two UM cell lines (92.1 and OMNI1) were assayed after treatment with varying concentrations of compound in combination with a fixed ASO 3 concentration (50 nM), which results in the selection of 7 compounds (FIGS. 2E-2K). Synergism was assessed using the excess over Bliss scores in both UM cell lines for both read outs (viability and confluence) at time point 72 h (FIG. 2D). All compounds are resulting in a positive (synergistic) excess over Bliss score in at least one cell line and for at least one read out. From the selected compounds, several are already FDA approved, such as amsacrine, mifepristone and clioquinol. The FDA approved compound amsacrine is a 9-aminoacridine derivative and clinically in use for the treatment of acute myeloid leukemia and malignant lymphomas²¹. Amsacrine intercalates into DNA, stabilizing the topoisomerase II-DNA complex resulting in double strand breaks²².

Other FDA approved compounds are mifepristone and clioquinol. Mifepristone, a progesterone receptor antagonist, is approved in obstetrics for early pregnancy termination²³. Mifepristone has also been linked to anti-tumor activity in multiple types of cancer and has been in clinical trials for the treatment of prostate cancer, with disappointing results^{23,24}. The other FDA approved drug, clioquinol, was used since the 1950's as an oral anti-parasitic agent for the treatment of fungal and protozoal infections. Due to sub-acute myelo-optic neuropathy (SMON), almost exclusively observed in Japanese patients, this drug was withdrawn from the market as an oral agent. Topical clioquinol remains in clinical use for certain fungal and skin disorders²⁵. Clioquinol also shows anti-cancer effects, probably due to the function as zinc ionophore, which, in melanoma cells, could be linked to mitochondrial swelling and loss of mitochondrial membrane potential²⁶.

Other drugs from the selection are still in clinical trials, such as CI-994 and entinostat, which are histone deacetylase (HDAC) inhibitors, resulting in hyperacetylation of histones and enabling gene transcription²⁷⁻²⁹. INH-6 is an inhibitor of the oncogene high expression in cancer 1 (Hec1) and its regulator, serine-threonine kinase Nek2, which together regulate mitotic spindle formation. Hec1 is overexpressed in many human cancers and inhibition of the Hec1/Nek2 pathway causes chromosome mis-alignment resulting in cell death^{30,31}. The final compound, GDC-0349, is a potent and selective inhibitor of mammalian target of rapamycin (mTOR), a serine/threonine kinase part of the phosphatidylinositol-3 (PI3K) kinase-related kinase (PIKK) family³². Also these compounds have already been reported to have in vitro and in vivo anti-tumor effects in multiple types of cancer^{28,30,32-34}.

### mTOR Inhibition Enhances SAMMSON ASO Activity In Vitro

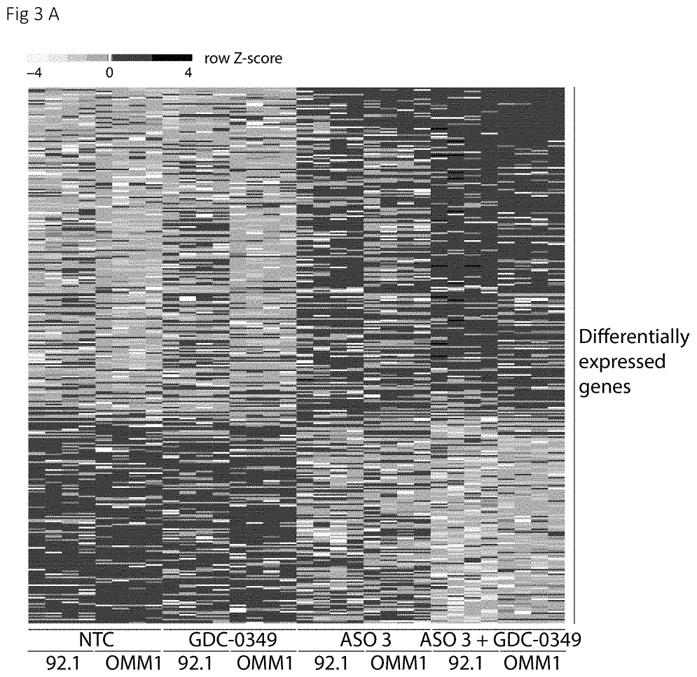

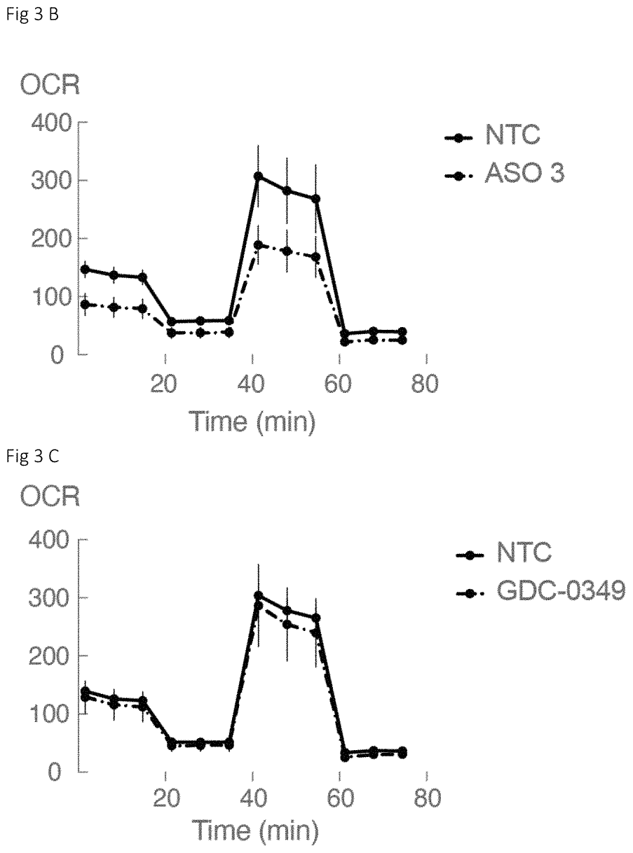

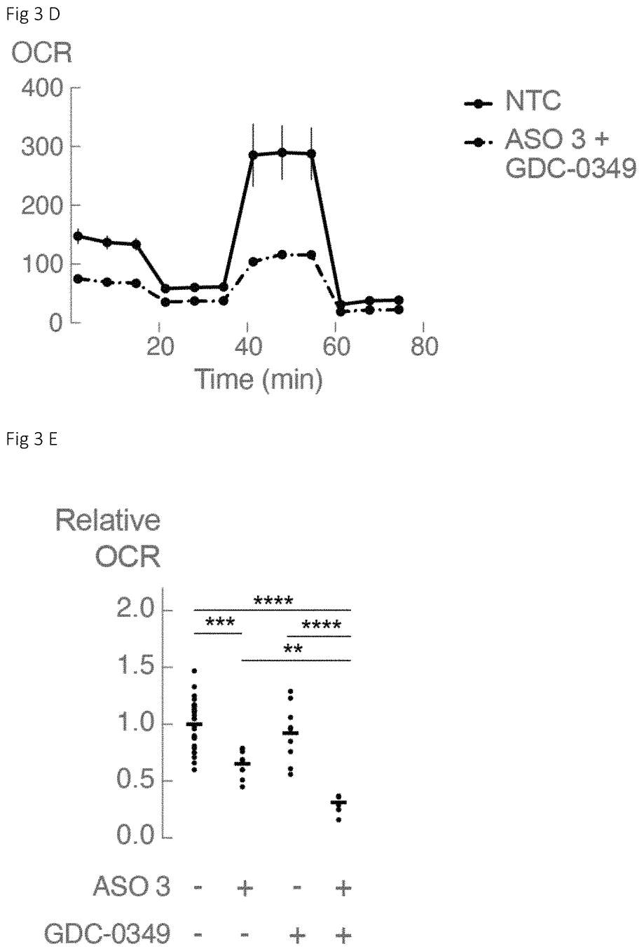

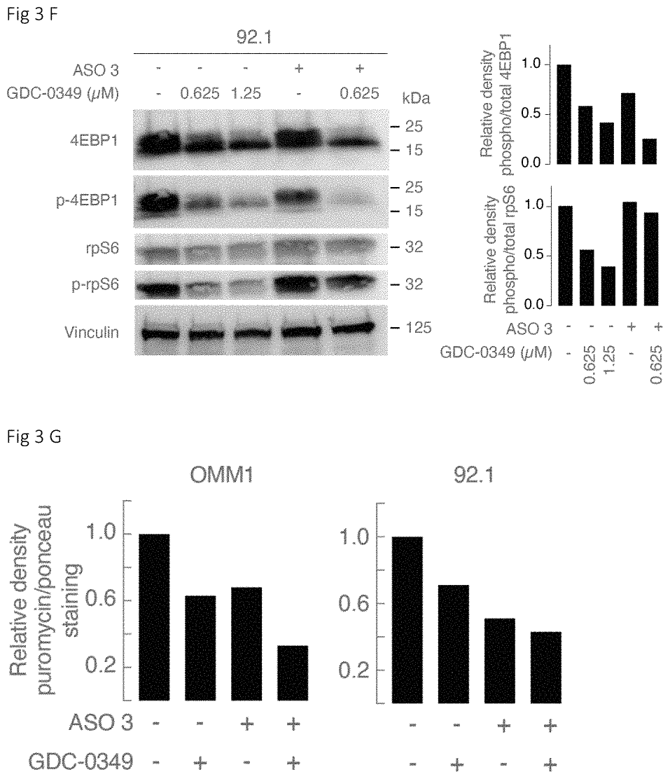

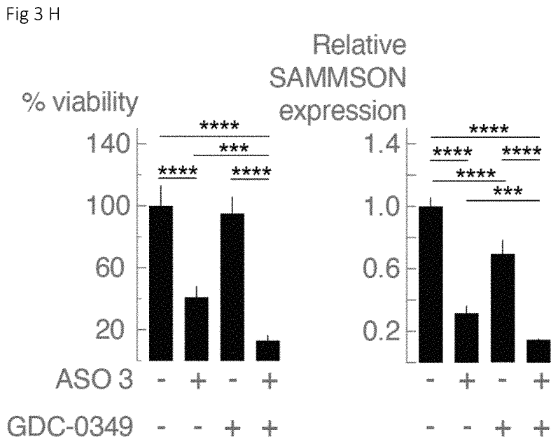

To investigate the contribution of GDC-0349 to obtain synergism, RNA sequencing was performed on UM cell lines 92.1 and OMM1 treated with either NTC ASO 50 nM or ASO 3 50 nM, in combination with either DMSO control or GDC-0349 0.625 04. Gene expression was measured 24 h after treatment. As illustrated in the heatmap showing all differentially expressed genes in at least one condition compared NTC ASO in both UM cell lines (92.1 and OMM1), GDC-0349 treated cells show a comparable pattern as NTC ASO, indicating GDC-0349 is not affecting the expression of many of these genes (FIG. 3A). In comparison to NTC ASO, ASO 3 reverses the expression pattern of the differentially expressed genes. Strikingly, when combining ASO 3 and GDC-0349, an enhanced reversal of the gene pattern can be observed. Some of the differentially expressed genes are involved in mitochondrial and translational processes, which confirms the contribution of SAMMSON in these pathways. In literature, it has been described that also mTOR affects the oxidative phosphorylation and mRNA translation in multiple cell lines³⁵. The mTOR inhibitor GDC-0349 at a concentration of 0.625 μM does not alter the mitochondrial function in UM cell line OMM1, while in combination with ASO 3 a significant impairment of the oxidative phosphorylation compared to single ASO 3 treatment can be observed (FIGS. 3B-3E). In addition, mTOR regulates protein synthesis by binding and phosphorylating eukaryotic initiation factor 4E-binding protein 1 (4E-BP1)) and ribosomal protein S6 kinase 1 (S6K1) of which the latter is part of the S6K family (S6K1 and S6K2)^{35,36}. Phosphorylated 4E-BP1 dissociates from eukaryotic initiation factor 4E (eIF4E), while phosphorylated S6K phosphorylates on its turn the ribosomal protein S6 (rpS6) on five C-terminal serine sites to allow full activation and consequently, initiating cap-dependent translation³⁷. Western blot confirms a dose dependent reduction of the phosphorylated rpS6 (S235/S236) and 4E-BP1 (T37/T46) levels by mTOR inhibition, while the total rpS6 and 4E-BP1 levels remain unaffected (FIG. 3F). Since SAMMSON also influences protein synthesis, the effect on the phosphorylation status of rpS6 and 4E-BP1 was investigated. SAMMSON inhibition results in less phosphorylated 4E-BP1 and in combination with mTOR inhibitor GDC-0349 hypo-phosphorylation goes beyond the individual effects (FIG. 3F). In contrast to 4E-BPI, SAMMSON inhibition doesn't affect the phosphorylation status of rpS6. Strikingly, upon ASO 3 and GDC-0349 treatment, the hypo-phosphorylation status of rpS6 due to GDC-0349 treatment is rescued to normal levels. Since 4E-BP1 and rpS6 are not the only regulators of protein translation, the effect on translation rate was investigated. SUnSET analysis confirms translation inhibition in UM cell lines 92.1 and OMM1 when treated with either GDC-0349 or ASO 3 (FIG. 3G). The combination of both further decreases the translation rate in both cell lines. Taken together, these results confirm mTOR is regulating translation by means of direct and indirect phosphorylation of 4E-BP1 and rpS6, respectively, while translational regulation by SAMMSON only partially occurs via the same pathway with phosphorylation of 4E-BP1.

Based on these results and recent research showing enhanced ASO-mediated target reduction upon mTOR inhibition³⁸, whether the GDC-0349 compound enhances the ASO activity was investigated. SAMMSON knock down was thus measured by combining ASO 3 treatment with GDC-0349, which resulted in an enhanced SAMMSON knockdown compared to single ASO 3 treatment (FIG. 3H). Together, these findings demonstrate that GDC-0349 enhances the ASO 3 uptake and activity in UM cells.

### REFERENCES

1. Bakalian, S. et al. Molecular Pathways Mediating Liver Metastasis in Patients with Uveal Melanoma. Clin. Cancer Res. 14, 951-956 (2008).

2. Papastefanou, V. P. & Cohen, V. M. L. Uveal Melanoma. Journal of Skin Cancer 2011, 1-13 (2011).

3. Rodríguez, A., Dueñas-Gonzalez, A. & Delgado-Pelayo, S. Clinical presentation and management of uveal melanoma. Mol Clin Oncol 5, 675-677 (2016).

4. Hoiom, V. & Helgadottir, H. The genetics of uveal melanoma: current insights. TACG Volume 9, 147-155 (2016).

5. Krantz, B. A., Dave, N., Komatsubara, K. M., Marr, B. P. & Carvajal, R. D. Uveal melanoma: epidemiology, etiology, and treatment of primary disease. OPTH Volume 11, 279-289 (2017).

6. Yan, X. et al. Comprehensive Genomic Characterization of Long Non-coding RNAs across Human Cancers. Cancer Cell 28, 529-540 (2015).

7. Verma, A. Neuromuscular Diseases: Recent Advances in Antisense Oligonucleotide Therapy. Astrocyte 5, 81-85 (2018).

8. Hong, D. et al. AZD9150, a next-generation antisense oligonucleotide inhibitor of STAT3 with early evidence of clinical activity in lymphoma and lung cancer. Sci Transl Med 7, 314ra185-314ra185 (2015).

9. Röhn, G. et al. ACTB and SDHA Are Suitable Endogenous Reference Genes for Gene Expression Studies in Human Astrocytomas Using Quantitative RT-PCR. Technol Cancer Res Treat 17, 153303381880231-6 (2018).

10. Burnichon, N. et al. SDHA is a tumor suppressor gene causing paraganglioma. Human Molecular Genetics 19, 3011-3020 (2010).

11. Aggarwal, A. et al. Optimal Reference Gene Selection for Expression Studies in Human Reticulocytes. The Journal of Molecular Diagnostics 20, 326-333 (2018).

12. Duan, J., Nilsson, L. & Lambert, B. Structural and functional analysis of mutations at the human hypoxanthine phosphoribosyl transferase (HPRT1) locus. Human Mutation 23, 599-611 (2004).

13. Aeola, F. Normalization of Gene Expression by Quantitative RT-PCR in Human Cell Line: comparison of 12 Endogenous Reference Genes. Ethiop J Health Sci 28, 1-8 (2018).

14. van Roon-Mom, W. M. C., Reid, S. J., Faull, R. L. M. & Snell, R. G. TATA-binding protein in neurodegenerative disease. Neuroscience 133, 863-872 (2005).

15. Sharan, R. N., Vaiphei, S. T., Nongrum, S., Keppen, J. & Ksoo, M. Consensus reference gene(s) for gene expression studies in human cancers: end of the tunnel visible? Cellular Oncology 1-13 (2015). doi:10.1007/s13402-015-0244-6

16. Kilic, E. et al. Clinical and Cytogenetic Analyses in Uveal Melanoma. Invest. Ophthalmol. Vis. Sci. 47, 3703-5 (2006).

17. Leucci, E. et al. Melanoma addiction to the long non-coding RNA SAMMSON. Nature 531, 518-522 (2016).

18. Yap, T. A., Omlin, A. & de Bono, J. S. Development of Therapeutic Combinations Targeting Major Cancer Signaling Pathways. JCO 31, 1592-1605 (2013).

19. Aggarwal, B. B., Danda, D., Gupta, S. & Gehlot, P. Models for prevention and treatment of cancer: Problems vs promises. Biochemical Pharmacology 78, 1083-1094 (2009).

20. Malo, N., Hanley, J. A., Cerquozzi, S., Pelletier, J. & Nadon, R. Statistical practice in high-throughput screening data analysis. Nat. Biotechnol. 24, 167-175 (2006).

21. Lee, Y.-C., Chen, Y.-J., Huang, C.-H. & Chang, L.-S. Amsacrine-induced apoptosis of human leukemia U937 cells is mediated by the inhibition of AKT- and ERK-induced stabilization of MCL1. Apoptosis 1-15 (2016). doi:10.1007/s10495-016-1307-5

22. Bin Zhang, Li, X., Bin Li, Gao, C. & Jiang, Y. Acridine and its derivatives: a patent review (2009 - 2013). Expert Opinion on Therapeutic Patents 24, 000-000 (2014).

23. Liu, R. et al. Mifepristone Suppresses Basal Triple-Negative Breast Cancer Stem Cells by Down-regulating KLF5 Expression. Theranostics 6, 533-544 (2016).

24. yang, Y., Li, X., Mamouni, K., Kucuk, O. & Wu, D. Mifepristone Has Limited Activity to Enhance the In Vivo Efficacy of Docetaxel and Enzalutamide Against Bone Metastatic and Castration-Resistant Prostate Cancer. AR 37, 6235-6243 (2017).

25. Ullah, A. Clioquinol-A Novel Copper-Dependent and Independent Proteasome Inhibitor. 1-7 (2011).

26. Ding, W.-Q., Liu, B., Vaught, J. L., Yamauchi, H. & Lind, S. E. Anticancer Activity of the Antibiotic Clioquinol. Cancer Res 1-8 (2005).

27. Hubeek, I. et al. CI-99 4 (N-acetyl-dinaline) in combination with conventional anti-cancer agents is effective against acute myeloid leukemia in vitro and in vivo. Oncol. Rep. 19, 1517-1523 (2008).

28. Prakash, S. et al. Chronic Oral Administration of CI-994: A Phase I Study. Invest New Drugs 19, 1-11 (2001).

29. Saito, A. et al. A synthetic inhibitor of histone deacetylase, MS-27-275, with marked in vivo antitumor activity against human tumors. PNAS 96, 4592-4597 (1999).

30. Qiu, X.-L. et al. Synthesis and Biological Evaluation of a Series of Novel Inhibitor of Nek2/Hec1 Analogues. J. Med. Chem. 52, 1757-1767 (2009).

31. Ferretti, C. et al. Expression of the kinetochore protein Hec1 during the cell cycle in normal and cancer cells and its regulation by the pRb pathway. Cell Cycle 9, 4174-4182 (2014).

32. Pei, Z. et al. Discovery and Biological Profiling of Potent and Selective mTOR Inhibitor GDC-0349. ACS Med. Chem. Lett. 4, 103-107 (2012).

33. Lee, J. et al. A class I histone deacetylase inhibitor, entinostat, enhances lapatinib efficacy in HER2-overexpressing breast cancer cells through FOXO3-mediated Bim1 expression. Breast Cancer Res Treat 146, 259-272 (2014).

34. Orillion, A. et al. Entinostat Neutralizes Myeloid-Derived Suppressor Cells and Enhances the Antitumor Effect of PD-1 Inhibition in Murine Models of Lung and Renal Cell Carcinoma. Clin. Cancer Res. 23, 5187-5201 (2017).

35. Morita,. et al. mTOR coordinates protein synthesis, mitochondrial activity and proliferation. Cell Cycle 14, 473-480 (2015).

36. Jossé, L., Xie, J., Proud, C. G. & Smales, C. M. mTORC1 signalling and eIF4E/4E-BP1 translation initiation factor stoichiometry influence recombinant protein productivity from GS-CHOK1 cells. Biochem. J. 473, 4651-4664 (2016).

37. Showkat, M., Beigh, M. A. & Andrabi, K. I. mTOR Signaling in Protein Translation Regulation: Implications in Cancer Genesis and Therapeutic Interventions. Molecular Biology International 2014, 1-14 (2014).

38. Ochaba, J. et al. A novel and translational role for autophagy in antisense oligonucleotide trafficking and activity. Nucleic Acids Res. 47, 11284-11303 (2019).

1. A pharmaceutical composition for treating a subject for uveal melanoma, the pharmaceutical composition comprising:
a. at least one inhibitor of functional expression of survival associated mitochondrial melanoma specific oncogene non-coding RNA (SAMMSON), which inhibitor(s) target(s) the SAMMSON gene or transcript directly by way of sequence complementarity and which is selected from the group consisting of a gapmer, an shRNA, an siRNA, an antisense RNA, a TALEN, and a Zinc-finger nuclease, and
b. at least one compound which is a topoisomerase II inhibitor, a mammalian target of rapamycin (mTOR) inhibitor, a HDAC inhibitor, a Hec1/Nek2 inhibitor, a progesterone receptor antagonist, and/or a zinc ionophore,
in amounts for treating the subject for uveal melanoma.

2. The pharmaceutical composition according to claim 1, wherein when the at least one compound is a topoisomerase II inhibitor, it is amsacrine, and/or wherein when the at least one compound is an mTOR inhibitor, it is GDC-0349 and/or wherein when the at least one compound is an HDAC inhibitor, it is entinostat and/or CI-994, and/or wherein when the at least one compound is an Hec1/Nek2 inhibitor, it is INH-6, and/or wherein when the at least one compound is a progesterone receptor antagonist, it is mifepristone, and/or wherein when the at least one compound is a zinc ionophore, it is clioquinol.

3. The pharmaceutical composition according to claim 2, wherein said inhibitor(s) of functional SAMMSON, which are locked nucleic acid antisense oligonucleotides having the following nucleic acid sequences: GTGTGAACTTGGCT (LNA ASO 3; SEQ ID NO:1) and TTTGAGAGTTGGAGGA (LNA ASO 11; SEQ ID NO:2).

4. A method of treating a subject for uveal melanoma, the method comprising:
administering to the subject a pharmaceutically effective amount of a composition comprising:
a. an inhibitor of functional expression of survival associated mitochondrial melanoma specific oncogene non-coding RNA (SAMMSON) which targets the SAMMSON gene or transcript directly by way of sequence complementarity and which is a gapmer, a shRNA, a siRNA, an antisense RNA, a TALEN, or a Zinc-finger nuclease, and
b. at least one compound which is a topoisomerase II inhibitor, a mammalian target of rapamycin (mTOR) inhibitor, a HDAC inhibitor, a Hec1/Nek2 inhibitor, a progesterone receptor antagonist, and/or a zinc ionophore.

5. The method according to claim 4, wherein when the at least one compound is a topoisomerase II inhibitor, it is amsacrine and/or wherein said when the at least one compound is an mTOR inhibitor, it is GDC-0349, and/or wherein when the at least one compound is an HDAC inhibitor, it is entinostat and/or CI-994, and/or wherein when the at least one compound is an Hec1/Nek2 inhibitor, it is INH-6, and/or wherein when the at least one compound is a progesterone receptor antagonist, it is mifepristone, and/or wherein when the at least one compound is a zinc ionophore, it is clioquinol.

6. The method according to claim 5, wherein said inhibitors of functional expression of SAMMSON, which are locked nucleic acid antisense oligonucleotides, have the following nucleic acid sequences: GTGTGAACTTGGCT (LNA ASO 3; SEQ ID NO:1) and TTTGAGAGTTGGAGGA (LNA ASO 11; SEQ ID NO:2).

7. The pharmaceutical composition of claim 1, wherein the at least one compound comprises amsacrine.

8. The pharmaceutical composition of claim 1, wherein the at least one compound comprises GDC-0349.

9. The pharmaceutical composition of claim 1, wherein the at least one compound comprises entinostat.

10. The pharmaceutical composition of claim 1, wherein the at least one compound comprises CI-994.

11. The pharmaceutical composition of claim 1, wherein the at least one compound comprises INH-6.

12. The pharmaceutical composition of claim 1, wherein the at least one compound comprises mifepristone.

13. The pharmaceutical composition of claim 1, wherein the at least one compound comprises clioquinol.

14. The method according to claim 4, wherein the at least one compound is a topoisomerase II inhibitor.

15. The method according to claim 4, wherein the at least one compound is an mTOR inhibitor.

16. The method according to claim 4, wherein the at least one compound is an HDAC inhibitor.

17. The method according to claim 4, wherein the at least one compound is an Hec1/Nek2 inhibitor.

18. The method according to claim 4, wherein the at least one compound is a progesterone receptor antagonist.

19. The method according to claim 4, wherein the at least one compound is a zinc ionophore.

20. A composition comprising:
a locked nucleic acid antisense oligonucleotide having SEQ ID NO:1,
a locked nucleic acid antisense oligonucleotide having SEQ ID NO: 2, and
at least one compound selected from the group consisting of amsacrine, GDC-0349, entinostat, CI-994, INH-6, mifepristone, and clioquinol.
